(19) United States
(12) Patent Application Publication
(10) Pub. No.: US 2021/0393834 A1
(43) Pub. Date: Dec. 23, 2021

(54) SYSTEMS AND METHODS FOR REDUCING DISEASE TRANSMISSION AMONG OCCUPANTS OF A BUILDING

(71) Applicant: Honeywell International Inc., Morris Plains, NJ (US)

(72) Inventor: Armin Wellig, Mont-sur-Rolle (CH)

(21) Appl. No.: 16/907,092

(22) Filed: Jun. 19, 2020

(51) Int. Cl. A61L 2/24; F24F 11/63; F24F 11/58; G05B 15/02; A61L 2/10; A61L 9/20

(52) U.S. Cl. CPC A61L 2/24; F24F 11/63; F24F 11/58; F24F 2120/10; A61L 2/10; A61L 9/20; G05B 15/02

(57) ABSTRACT

A system includes one or more occupancy sensors, one or more environmental sensors and one or more controllable building components. A controller is configured to receive occupancy signals from the one or more occupancy sensors over a building network and to receive indoor air quality parameter signals from the one or more environmental sensors over the building network and to process the received signals to determine whether action is needed to improve one or more environmental conditions within at least some of the plurality of building spaces to reduce the likelihood of disease transmission among occupants of the building. Responsive to determining that action is needed, the controller is configured to provide control signals to one or more of the controllable building components over the building network to improve one or more environmental conditions within at least some of the plurality of building spaces.

### TECHNICAL FIELD

The present disclosure relates generally to facility management systems, and more particularly to systems and methods for reducing risk of disease spread among occupants of a building.

### BACKGROUND

Infectious diseases can spread through person to person contact, touching of contaminated surfaces, exposure to air borne pathogens, as well as other transmission mechanisms. What would be desirable are systems and methods to help limit the spread of a disease among occupants of a building.

### SUMMARY

The present disclosure relates generally to tracking and disinfection spaces within a building. In an example, a system reduces the likelihood of disease transmission among occupants of a building that has a plurality of building spaces. The system includes one or more occupancy sensors that are positioned to detect occupancy within each of the plurality of building spaces. Each of the one or more occupancy sensors are operably coupled with a building network and provide occupancy signals over the building network. One or more environmental sensors such as indoor air quality sensors are positioned to detect one or more environmental parameters within at least some of the plurality of building spaces. Each of the one or more environmental sensors are operably coupled with the building network and can provide environmental parameter signals over the building network. The system includes one or more controllable building components for controlling environmental conditions within at least some of the plurality of building spaces to reduce the likelihood of disease transmission among occupants of the building, the one or more controllable building components controllable over the building network. A controller is operably coupled with the building network and is configured to receive occupancy signals from the one or more occupancy sensors over the building network and to receive environmental parameter signals from the one or more environmental sensors over the building network. The controller is configured to process the received occupancy signals and the received environmental parameter signals to determine whether action is needed to improve one or more environmental conditions within at least some of the plurality of building spaces to reduce the likelihood of disease transmission among occupants of the building. Responsive to determining that action is needed, the controller is configured to control signals to one or more of controllable building components over the building network to improve one or more environmental conditions within at least some of the plurality of building spaces to reduce the likelihood of disease transmission among occupants of the building.

In another example, a method maintains a level of occupant safety within a building that has a building space. The building space includes an occupancy sensor and one or more air quality sensors and is serviced by a heating, ventilating and air conditioning (HVAC) system. Each of the sensors and the HVAC system are operably coupled with a building network. Occupancy signals are received from the occupancy sensor and indoor air quality parameter signals are received from the one or more indoor air quality sensors. The occupancy signals and the indoor air quality parameter signals are processed to determine whether action is needed to maintain the level of safety within the building space. Responsive to determining that action is needed, control signals are outputted to the HVAC system via the building network.

In another example, a system reduces pathogenic exposure within a building having a plurality of building spaces. The system includes one or more occupancy sensors that are positioned to detect occupancy within each of the plurality of building spaces, each of the one or more occupancy sensors operably coupled with a building network. The system includes one or more sanitizers, each of the one or more sanitizers positioned to sanitize surfaces within a corresponding one of the plurality of building spaces. A controller is operably coupled with the building network and is configured to receive occupancy signals from the one or more occupancy sensors and to process the occupancy signals to determine whether a particular building space is due to be sanitized and is currently available to be sanitized. Responsive to determining that the particular building space is due to be sanitized and is currently available to be sanitized, appropriate control signals are outputted to one or more of the one or more sanitizers to proceed with sanitizing the particular building space. In some instances, a single system may provide both occupancy safety while reducing pathogenic exposure, depending on whether the space is occupied or not.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE FIGURES

The disclosure may be more completely understood in consideration of the following description of various examples in connection with the accompanying drawings, in which:

FIG. 1 is a schematic block diagram of an illustrative building management system;

FIG. 2 is a schematic block diagram of an illustrative building management system;

FIG. 3 is a schematic block diagram of an illustrative building management system;

FIG. 4 is a schematic block diagram of an illustrative building management system;

FIG. 5 is a schematic block diagram of an illustrative building management system;

FIG. 6 is a flow diagram showing an illustrative method that may be carried out via the illustrative building management systems of FIGS. 1 through 5;

FIG. 7 is a flow diagram showing an illustrative method that may be carried out via the illustrative building management systems of FIGS. 1 through 5; and

FIG. 8 is a schematic block diagram illustrating relationships between sensing and corresponding actions.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic is described in connection with an embodiment, it is contemplated that the feature, structure, or characteristic may be applied to other embodiments whether or not explicitly described unless clearly stated to the contrary.

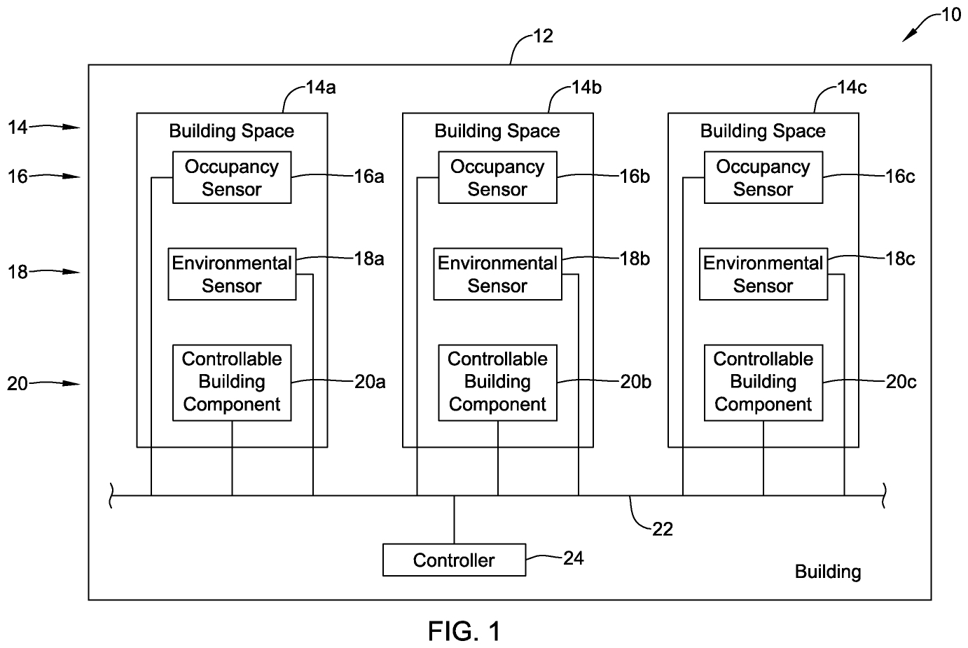

FIG. 1 is a schematic block diagram of an illustrative building management system 10. The illustrative building management system 10 is installed in a building 12 and may be considered as being configured to reduce the risk of pathogenic exposure within the building 12. The building 12 includes a number of building spaces 14 that are individually labeled as 14a, 14b, 14c. It will be appreciated that this is merely illustrative, as the building 12 will typically include a much greater number of building spaces 14 or zones. At least some of the building spaces 14 may periodically have one or more people within the building space 14. In some cases, the building 12 may be a hotel, and thus the building spaces 14 may be individually rentable guest rooms. The building 12 may be an office building, or a portion of an office building, and thus the building spaces 14 may be individual offices or work spaces. In some case, the disclosure may be applied to a cruise ship.

Each of the building spaces 14 includes one or more occupancy sensors 16, although only one occupancy sensor 16 is shown in each of the building spaces 14. The occupancy sensors 16 are individually labeled as 16a, 16b, 16c. At least some of the occupancy sensors 16 may be PIR sensors, mmWave sensors, motion sensors and/or microphones, for example. Some of the occupancy sensors 16 may be part of a security system of the building 12, for example. In some cases, some of the occupancy sensors 16 may be video cameras that are coupled with video analytics to detect the presence of one or more people, and hence determine occupancy. Occupancy detection may include detecting the presence of people, including counting people. Occupancy detection may also include behavioral indicators such as hand washing, signs of illness such as fever and coughing, spacing between people and the like.

Each of the building spaces 14 also include one or more environmental sensors 18, although only one environmental sensor 18 is shown in each of the building spaces 14. The environmental sensors 18 are individually labeled as 18a, 18b, 18c. The environmental sensors 18 may, for example, be sensors such as temperature sensors, humidity sensors, visible light sensors, UV sensors, particulate matter sensors (e.g. PM2.5, PM10), VOC sensors, airborne and waterborne pathogen sensors, CO sensors, CO2 sensors, ozone sensors, and/or any other environmental suitable sensor. In some cases, some of the environmental sensors 18 may be considered as being Indoor Air Quality (IAQ) sensors. In some cases, one or more of the environmental sensors 18 may be disposed within a room thermostat within at least some of the building spaces 14.

In some cases, sensing environmental parameters may include sensing air pressure in general, and air pressure differentials across the building 12 in particular. It has been found that air pressure differentials can provide a general indication of air flow through the building 12. Air fill flow from an area of higher pressure to an area of lower pressure, for example. Measuring air pressure differentials can also provide an indication of how opening and closing windows and doors can influence air flow through the building 12, for example. Measuring air pressure differentials can also provide an indication of the impact of turning ventilation on or off, or turning ventilation rates up and down, among other HVAC capabilities. In some cases, controlled air flow is one of the key techniques highlighted by ASHRAE (American Society of Heating, Refrigerating and Air Conditioning Engineers) to control airborne pathogen transmission.

If a building space 14 is in a hospital operating room, for example, there is a desire to maintain an air pressure within the operating room that is higher than the air pressure in neighboring spaces. This can help to limit airborne pathogens from entering the operating room, as any air movement will tend to be from inside the operating room to outside of the operating room. If a building space 14 is not occupied, there may be a desire to reduce air flow in the duct(s) that provide conditioned air to the building space 14 in order to increase an amount of time that any airborne pathogens are exposed to UV light during a sanitizing process. These are just examples.

Each of the building spaces 14 includes one or more controllable building components 20, although only one controllable building component 20 is shown in each of the building spaces 14. Each of the controllable building components 20 may be considered as being configured to control environmental conditions within the building spaces 14 in order to reduce the likelihood of disease transmission among occupants of the building 12. At least some of the controllable building components 20 may include heating, ventilating and air conditioning system (HVAC) components such as heating sources, cooling sources, ventilation sources, humidifiers and dehumidifiers, as examples. At least some of the controllable building components 20 may include a disinfecting component. Examples of disinfecting components include sources of UV light that may be used to sanitize surfaces within the building space 14. UV light sources may also be used to disinfect components of an HVAC system, such as but not limited to disinfecting filters within the HVAC system. This may include cleaning filter media as well as electrostatic filters.

The UV light spectrum ranges from about 100 nanometers (nm) to about 400 nm. The UV light spectrum includes UV-A, which ranges from 315 nm to 400 nm. This UV light spectrum also includes UV-B, which ranges from 280 nm to 315 nm. UV-C, which ranges from 200 nm to 280 nm, is particularly effective for disinfecting. There is also Far-UVC, which ranges from 207 nm to 222 nm and thus is a subset of the UV-C light spectrum. Far-UVC is also particularly effective for disinfecting, and is believed to be safe for human skin and eyes. The UV light spectrum also includes VUV Far-UV, which ranges from 100 nm to 200 nm. In some cases, at least some of the controllable building components 20 may include a source of UV-C light that is configured to provide UV-C light for a period of time sufficient to disinfect surfaces within the building space 14. For example, it may take a period of time, such as 5 minutes, 10 minutes, 30 minutes, 1 hour, 2 hours, 3 hours or more, depending on a number of factors such as the intensity of the UV-C light source and the distance between the UV-C light source and the surfaces to be sanitized.

In the example shown, each of the occupancy sensors 16, each of the environmental sensors 18 and each of the controllable building components 20 are operably coupled with a building network 22. A controller 24 is operably coupled with the building network 22 such that the controller 24 is able to receive occupancy data from the occupancy sensors 16 and indoor air quality data from the environmental sensors 18. Accordingly, each of the one or more occupancy sensors 16 may be considered as providing occupancy signals over the building network 22. Similarly, each of the one or more environmental sensors 18 may be considered as providing air quality parameter signals over the building network 22. In some cases, the one or more environmental sensors 18 may provide a measurement of carbon dioxide concentration as a basic occupancy indicator. It will be appreciated that carbon dioxide concentration will increase as additional people are present within the building space 14, and will decrease as people leave the building space 14.

The controller 24 is also able to provide control signals to the controllable building components 20 via the building network 22. It is contemplated that the building network 22 may be a wired network, a wireless network or a combination of wired and wireless. It will be appreciated that while the controller 24 is shown as being located inside of the building 12, this is not required in all cases. In some instances, the controller 24 may itself be manifested within one or more computing devices that may be local to the building 12 or may be remote from the building 12. In some case, all or part of the controller 24 may be manifested within a cloud-based server.

In some instances, the controller 24 is configured to receive occupancy signals from the one or more occupancy sensors 16 over the building network 22 and to receive indoor air quality parameter signals from the one or more environmental sensors 18 over the building network 22. The controller 24 is configured to process the received occupancy signals and the received indoor air quality parameter signals (sometimes in combination) to determine whether action is needed to improve one or more environmental conditions within at least some of the plurality of building spaces 14 in order to reduce the likelihood of disease transmission among occupants of the building 12. Responsive to determining that action is needed, the controller 24 is configured to send control signals to one or more of controllable building components over the building network to improve one or more environmental conditions within at least some of the plurality of building spaces to reduce the likelihood of disease transmission among occupants of the building.

In some instances, the controller 24 is configured to process one or more of the received occupancy signals to identify a measure of compliance of one or more of the occupants of the building 12 with one or more predefined behavioral standards. In some cases, the occupancy signals may include locating sensors that can report a location of the one or more occupants of the building. The locating sensors may include, for example, Bluetooth or WiFi beacons that can be used to track an occupants phone's location in the building. It is contemplated that the locating sensor may be any locating sensor or system that is can identify a location of the one or more occupants in the building. In some cases, the occupancy signals may include video signals from a video camera or signals from an indoor radar sensor (e.g. mmWave sensor) that can be used to identify compliance of one or more of the occupants of the building 12 with one or more predefined behavioral standards. These are just examples.

One example of a predefined behavioral standard includes a social distancing standard. In some instances, maintaining a minimum distance between people may help prevent the transmission of disease. Social distancing can help reduce or limit the spread of disease from both symptomatic and asymptomatic carriers. The social distancing standard may be set to a particular physical distance that individuals should strive to maintain between themselves and other individuals, and may be determined at least in part upon the particular environment. People produce aerosols when talking, breathing, coughing and the like. If people stay far enough apart, these aerosols are able to drop out of the air before they reach another person who could inhale the aerosol and become infected. Relative humidity can impact how far an aerosol can travel. As an example, a social distancing standard may be set equal to 6 feet, or 12 feet.

Another example of a predefined behavioral standard includes a maximum people per building space standard. This may be equivalent to the maximum capacity for a particular building space 14, such as dictated by fire code. The maximum people per building space standard may dictate a reduced maximum capacity, particularly during times in which a particular pathogen is believed to be active. For example, the maximum people per building space standard during times in which pathogens are active may be set equal to fifty percent of the maximum occupancy dictated by fire code. The maximum people per building space standard may be set equal to twenty five percent the maximum occupancy dictated by fire code. These are just examples, as these numbers may vary depending on the communicability of a particular pathogen, the susceptibility of the people within the building 12 to the particular pathogen, and the like.

As another example, a predefined behavioral standard may include a hygiene standard. Examples of hygiene standards include whether individuals are wearing masks, particularly if the people are inside the building 12 and are not always complying with social distancing standards. Hygiene standards may also dictate whether a mask is required based upon how long two people may be in proximity to each other, as length of exposure can influence the degree or likelihood of pathogen transmission. The longer a person is exposed to a carrier, for example, the more of the pathogen that the person will have likely been exposed to and thus may now possess. Another example of a hygiene standard involves hand washing. This may be as simple as requiring that people wash their hands after using the bathroom. This may also include requiring people to wash their hands each time they move from one space to another space, or are about to eat or drink something, for example. Hand washing standards may specify a duration of time that hand washing is expected to last in order to be effective. For example, a hand washing time of twenty seconds is contemplated. Hygiene standards may also include specifying whether people are wearing gloves, as well as details regarding when, where and how the person is expected to wear gloves. A predefined behavioral standard may include ascertaining compliance with hygiene standards such as but not limited to wearing gloves, wearing masks, and hand washing.

In some instances, a predefined behavioral standard may include a symptoms standard. Depending on a suspected pathogen or a resulting disease, it will be appreciated that symptomatic individuals with the suspected pathogen or disease may have any of a variety of different symptoms. For example, various respiratory diseases may have symptoms that include coughing and/or sneezing. An elevated body temperature, better known as a fever, is also a common disease symptom as in many cases, the elevated body temperature is the result of the person's immune system trying to fight off the pathogen.

In some instances, a predefined behavioral standard may include a cleaning standard that is associated with a cleaning crew. This may include, for example, whether the correct area gets cleaned when that area is supposed to get cleaned. This may include whether the actual duration of the cleaning process meets or exceeds a cleaning time standard for that particular building space, for that particular cleaning crew and the like. Cleaning standards may also refer to automated cleaning and disinfecting processes, such as but not limited to an expected duration for exposing a surface or surfaces to UV-C light, for example. A disinfecting process may be set to last a particular length of time, but can be interrupted if a door is opened prematurely, for example. In some instances, the sensing controls may be integrated into a workflow application in order to provide a cleaning score or map that provides real-time insights to the cleaning staff. This may facilitate the cleaning staff being able to identify and take care of any missed spots, for example.

The controller 24 may be configured to process one or more of the received occupancy signals to identify a measure of a total number of occupants within one or more of the plurality of building spaces 14 of the building 12, and may use the measure of the total number of occupants within one or more of the plurality of building spaces 14 of the building 12 to determine whether action is needed. In some cases, the controller 24 may be configured to determine when action is needed by one or more persons to improve one or more environmental conditions within at least some of the plurality of building spaces 14 to reduce the likelihood of disease transmission among occupants of the building 12, and provide a notification. In some cases, the controller 24 may be configured to track occupancy over time, and thus may be able to learn when particular building spaces 14 are expected to remain empty.

In some instances, the controller 24 may be configured to determine that action is needed to maintain the indoor air quality when one or more indoor air quality parameters exceed a threshold for the corresponding one or more indoor air quality parameters, and in response, the controller 24 may be configured to output appropriate control signals requesting, for example, an increase in fresh air to the building space 14, an increase or decrease in a temperature and/or humidity level, and/or any other suitable change to the environment.

In some cases, the controller 24 may be configured to determine that action is needed to maintain the indoor air quality when one or more of the building spaces 14 have an indicated occupancy that is above a first occupancy threshold, and in response, the controller 24 may be configured to output appropriate control signals requesting, for example, an increase in fresh air to the building space 14, an increase or decrease in a temperature and/or humidity level, and/or any other suitable change to the environment. The controller 24 may be configured to determine that action is needed to maintain the indoor air quality when one or more of the building spaces 14 have an indicated occupancy that is at a second occupancy threshold, and in response, the controller 24 may be configured to output appropriate control signals indicating that no additional people are permitted in the one or more building spaces. The control signals may include instructions for an audio and/or visual warning that occupancy has reached the second threshold, and/or instructions to lock one or more doors in order to prevent additional people from entering the one or more building spaces.

In some instances, the controller 24 may be configured to process one or more of the received indoor air quality parameter signals to identify a measure of compliance with one or more air quality standards. Examples air quality standards include a relative humidity standard and a temperature standard. Other examples of air quality standards include a carbon dioxide (CO₂) standard, a carbon monoxide (CO) standard, a Particulate Matter (PM) standard, a pathogen concentration standard, a Volatile Organic Compound (VOC) standard, a H2CO standard.

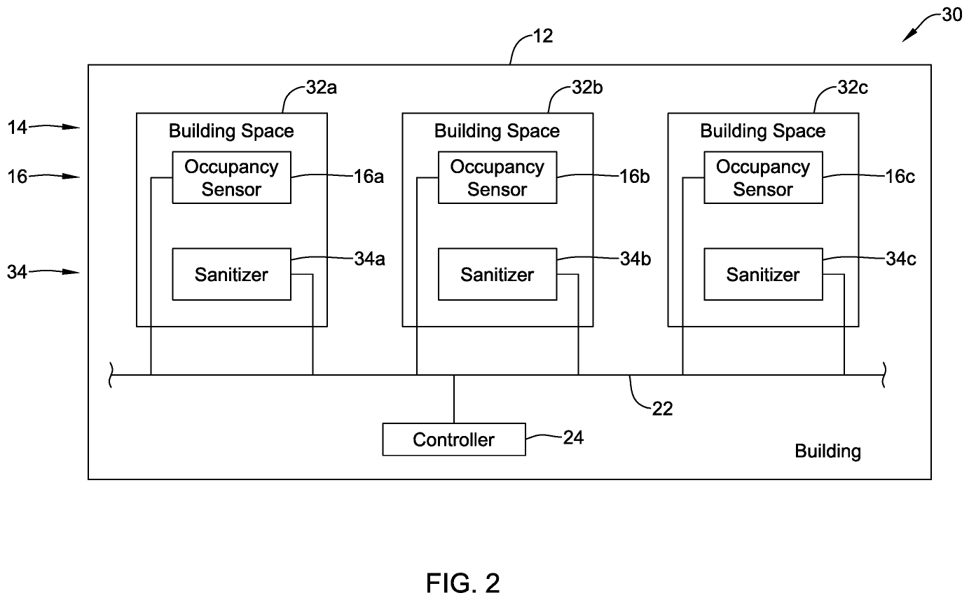

FIG. 2 is a schematic block diagram of an illustrative building management system 30. The illustrative building management system 30 is shown as being installed in the building 12 and may be considered as being configured to reduce the risk of pathogenic exposure within the building 12. The building 12 includes a number of building spaces 32 that are individually labeled as 32a, 32ab, 32c. It will be appreciated that this is merely illustrative, as the building 12 will typically include a much greater number of building spaces 32 or zones. At least some of the building spaces 32 may periodically have one or more people within the building space 32. In some cases, at least some of the building spaces 32 may be considered as being examples of the building spaces 14. At least some of the building spaces 32 may include features ascribed to the building spaces 14. At least some of the building spaces 14 may include features ascribed to the building spaces 32.

In addition to the occupancy sensors 16, each of the building spaces 32 include one or more sanitizers 34, although only one sanitizer 34 is shown in each of the building spaces 32. The sanitizers are individually labeled as 34a, 34b, 34c. In some cases, the sanitizers 34 may each be sources of UV-C light. The sanitizers 34 may be considered as being positioned to sanitize surfaces within a corresponding building space 32. Disinfecting with other processes such as plasma and ionization is also contemplated. Each of the one or more occupancy sensors 16 may be considered as being positioned to detect occupancy within each of the plurality of building spaces 32, and may be considered as being operably connected to the building network 22. The controller 24 is configured to receive occupancy signals from the one or more occupancy sensors 16 and to process the occupancy signals to determine whether a particular building space 32 is due to be sanitized and is currently available to be sanitized. In some cases, determining whether the particular building space 32 is due to be sanitized is based at least in part upon how long it has been since that particular building space 32 was last sanitized and/or how many people have been in that particular building space 32 since it was last sanitized. In response to determining that the particular building space 32 is due to be sanitized and is currently available to be sanitized, the controller 24 outputs appropriate control signals to one or more of the one or more sanitizers 34 to proceed with sanitizing the particular building space 32.

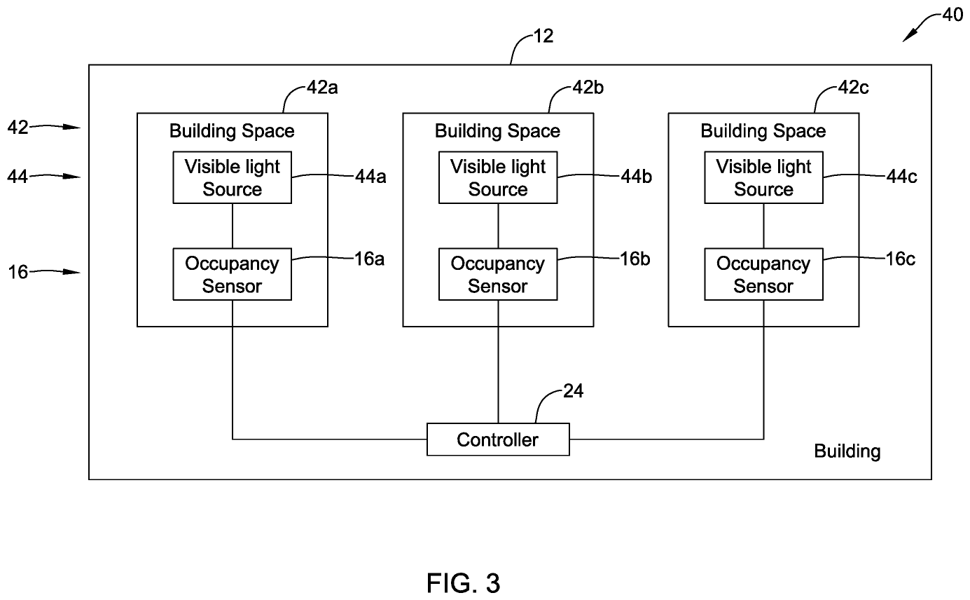

FIG. 3 is a schematic block diagram of an illustrative building management system 40. The illustrative building management system 40 is shown as being installed in the building 12 and may be considered as being configured to disinfect spaces within the building 12. The building 12 includes a number of building spaces 42 that are individually labeled as 42a, 42ab, 42c. It will be appreciated that this is merely illustrative, as the building 12 will typically include a much greater number of building spaces 42 or zones. At least some of the building spaces 42 may periodically have one or more people within the building space 42. In some cases, at least some of the building spaces 42 may be considered as including features ascribed to the building spaces 14 and/or the building spaces 32. At least some of the building spaces 14 and 32 may include features ascribed to the building spaces 42.

In addition to the occupancy sensors 16, each of the building spaces 42 include one or more visible light sources 44, although only one visible light source 44 is shown in each of the building spaces 42. The visible light sources 44 are individually labeled as 44a, 44b, 44c. The visible light sources 44 may be considered as being distributed throughout each of at least some of the plurality of building spaces 42 of the building 12, wherein each of the plurality of visible light sources 44 is associated with a corresponding occupancy sensor 16, wherein when occupancy is detected, the corresponding visible light source 44 is turned on, and when occupancy is not detected, the corresponding visible light source 44 is turned off. In some instances, some of the occupancy sensors 16 may be integrated into a corresponding visible light source 44.

In some instances, the controller 24 may be configured to monitor the occupancy sensors 16 to infer an occupancy distribution in each of the at least some of the plurality of building spaces 42 of the building 12 and to compare the occupancy distribution in each of the at least some of the plurality of building spaces 42 of the building 12 with a corresponding occupancy distribution threshold. The controller 24 may be configured to take action in response to determining that the occupancy distribution in one or more of the at least some of the plurality of building spaces 42 of the building 12 exceeds the corresponding occupancy distribution threshold. Taking action may, for example, include providing an audio and/or visual warning that the occupancy distribution in one or more of the at least some of the plurality of building spaces 42 of the building 12 exceeds the corresponding occupancy distribution threshold. This is just an example. In some cases, the occupancy distribution threshold of one of the plurality of building spaces 42 may be different from the occupancy distribution threshold of another one of the plurality of building spaces 42.

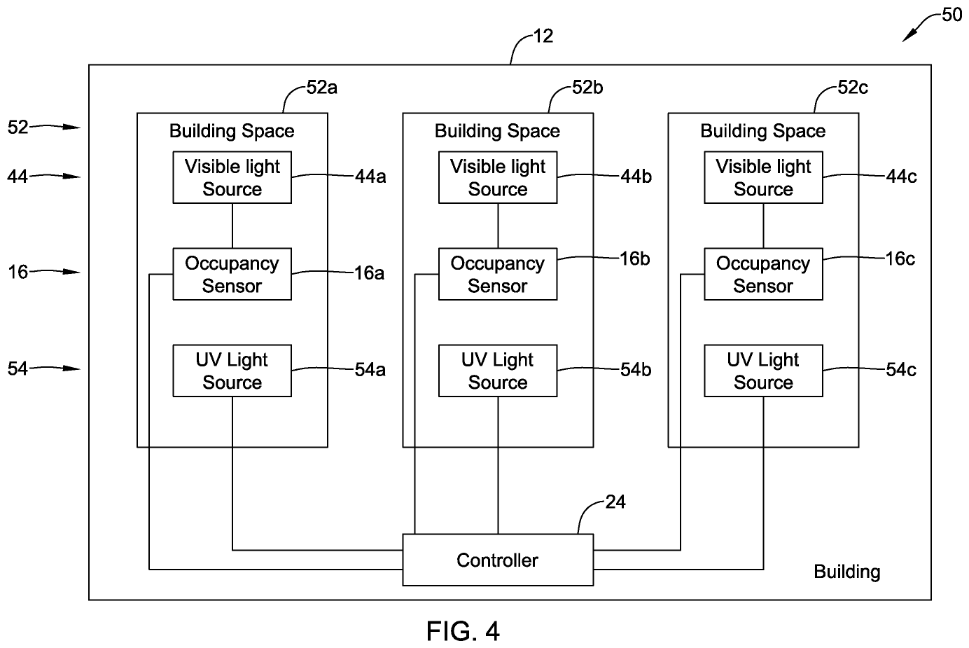

FIG. 4 is a schematic block diagram of an illustrative building management system 50. The illustrative building management system 50 is shown as being installed in the building 12 and may be considered as being configured to disinfect spaces within the building 12. The building 12 includes a number of building spaces 52 that are individually labeled as 52a, 52ab, 52c. It will be appreciated that this is merely illustrative, as the building 12 will typically include a much greater number of building spaces 52 or zones. At least some of the building spaces 52 may periodically have one or more people within the building space 52. In some cases, at least some of the building spaces 52 may be considered as including features ascribed to the building spaces 14 and/or the building spaces 32 and/or the building spaces 42. At least some of the building spaces 14, 32 and 42 may include features ascribed to the building spaces 52.

In addition to the visible light sources 44 and the occupancy sensors 16, each of the building spaces 52 includes one or more UV light sources 54, although only one UV light source 54 is shown within each of the building spaces 52. The UV light sources 54 may be UV-C light sources, for example. The UV light sources 54 are individually labeled as 54a, 54b, 54c. Each of the visible light sources 44 are associated with a corresponding occupancy sensor 16 such that when occupancy is detected (by a particular occupancy sensor 16) the corresponding visible light source 44 is turned on, and when occupancy is not detected, the corresponding visible light source 44 is turned off.

The controller 24 is operably coupled to at least some of the occupancy sensors 16 and the plurality of UV light sources 54. In some instances, the controller 24 is configured to monitor the occupancy sensors 16 to determine when one or more of the plurality of building spaces 52 of the building 12 are unoccupied. The controller 24 may activate one or more of the UV light sources 54 in one or more of the plurality of building spaces 52 of the building 12 that are determined to be unoccupied. In some instances, the controller 24 may further determine when one or more of the plurality of building spaces 52 of the building 12 are unoccupied and are expected to remain unoccupied for at least a predetermined period of time based at least in part on a historical occupancy pattern.

In some cases, the controller 24 may activate the one or more UV light sources 54 for a predetermined period of time. The predetermined period of time may be user-adjustable, for example, by building management for the building 12. The predetermined period of time may be set when the system 50 is initially configured. The controller 24 may monitor the occupancy sensors 16 to determine if one or more of the plurality of building spaces 52 of the building 12 where the one or more of the UV light sources 54 were activated become re-occupied, and if so, deactivating the corresponding UV light sources 54. This can help prevent an individual walking into the building space 52 from being exposed to possibly damaging UV light. In some instances, the building space 52 may include a UVC sensor (not shown) that provides additional feedback to the controller 24 that an active UV light source is present, such that the controller 24 can provide a warning that the UV light source is active, and that the controller 24 can shut off the UV light source if occupancy is detected. In some cases, a signal from the UVC sensor can be taken into account when tracking disinfection statistics including how frequently and/or the duration of disinfecting processes.

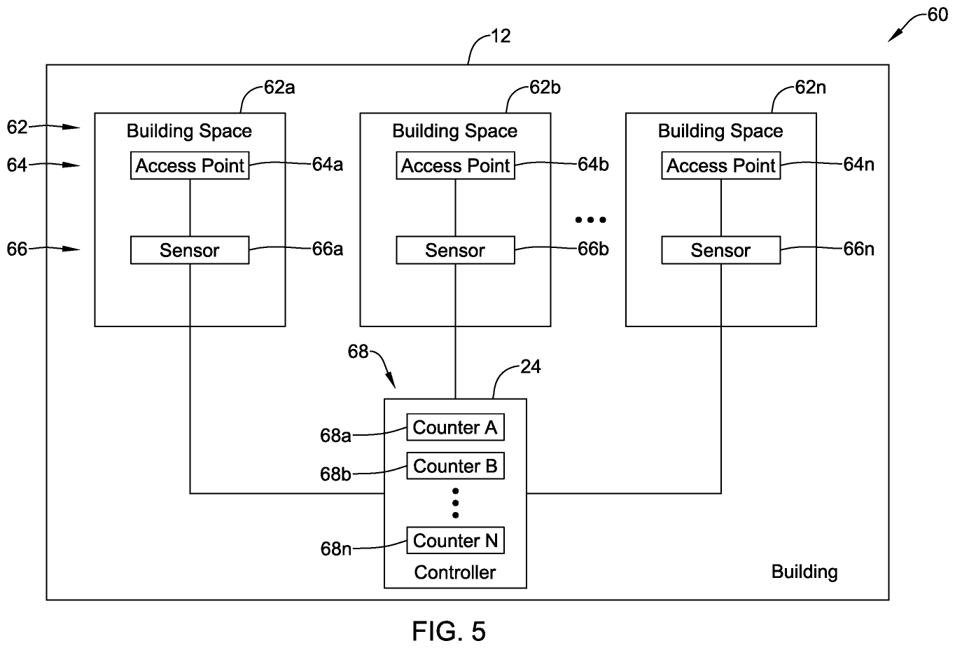

FIG. 5 is a schematic block diagram of an illustrative building management system 60. The illustrative building management system 60 is shown as being installed within the building 12. The building 12 includes a number of building spaces 62 that are individually labeled as 62a, 62ab through 62n. At least some of the building spaces 62 may periodically have one or more people within the building space 62. In some cases, at least some of the building spaces 62 may be considered as including features ascribed to the building spaces 14 and/or the building spaces 32 and/or the building spaces 42 and/or the building spaces 52. At least some of the building spaces 14, 32, 42 and 52 may include features ascribed to the building spaces 62.

Each building space 62 includes at least one access point 64, although only one access point 64 is shown within each of the building spaces 62. The access points 64 are individually labeled as 64a, 64b through 64n. In some cases, a building space 62 may have more than one access point 64. In simplest terms, the access point 64 may be a physical or virtual door that allows people to enter and/or exit the building space 62. In some cases, the physical or virtual door can be closed to prevent additional people from passing through the access point 64 and entering the building space 62. An access point 64 may be a designated region of a hallway, elevator, lobby or the like, that can be considered as providing access to a particular building space 62.

Each building space 62 includes at least one sensor 66, although only one sensor 66 is shown in each of the building spaces 62. The sensors 66 are individually labeled as 66a, 66b through 66n. In some cases, there may be one sensor 66 that is associated with each of the access points 64. The sensors 66 may be configured to output a signal that indicates that someone has passed through an access point 66, either entering the building space 62 or exiting the building space 62. In some cases, at least some of the sensors 66 may include smart floor sensors that a person walks across. As the person walks across the smart floor sensor, a pressure signal is outputted that can indicate a direction of travel (i.e., into the building space 62 or out of the building space 62). The pressure signal may, for example, enable analysis that yields a shape or transient characteristics of a pressure input made by the person's foot, and thus a direction of travel can be determined. The smart floor sensor(s) can be embedded in a floor covering, such as but not limited to an area rug or mat. Thus, they can easily be used in a hallway to define an access point 64 (and corresponding sensor 66). In some cases, the smart floor sensor is large enough that a person passing through the access point 64 has to take multiple steps crossing the smart floor sensor. As a result, the direction of their travel is easily determined.

In some cases, at least some of the sensors 66 may include passive infrared (PIR) sensors. By placing two or more PIR sensors in the building space 62, it is possible to determine a direction of travel for a person passing through the access point 64. In some instances, at least some of the sensors 66 may include augmented PIR sensors that are configured to provide output signals that can indicate one or more of direction, distance and speed of a person traveling through the access point 64. In some cases, a building space 62 may include both a smart floor sensor and a PIR sensor as the sensors 66, such that a signal from one of the sensors 66 may be used as a check or confirmation of a signal from the other of the sensors 66. In some cases, at least some of the sensors 66 may include RFID card readers that can read an access card carried by a person without requiring the person to physically scan the access card. One or more of the sensors 66 may include a time of flight sensor that is based on a laser bean interruption, or a video camera. These are just some examples. With these and other types of sensors, the occupants can be granted frictionless access to a particular building space 62.

In some cases, Time of Flight (ToF) may be implemented as a technology that can count people moving through the access point 64. ToF can provide accurate people counting at low to medium traffic density, such as may occur when several people walk into a room at around the same time. ToF involves creating a pair of sensor zones, such as a front zone and a back zone. A person is detected as they cross through both the front zone and the back zone. In some instances, the sensors used for ToF measurements may be placed behind an optically opaque polymer panel. This can mean that ToF can be implemented unobtrusively, without people realizing that they are walking past a sensor, or even realizing that they are being counted.

In some instances, the controller 24 maintains a counter 68 for each of the access points 64. The controller 24 may include a counter 68a that corresponds to the sensor 66a, a counter 68b that corresponds to the sensor 66b, all the way through a counter 68n that corresponds to the sensor 66n. The appropriate counter 68 may be incremented in response to the controller 24 receiving an indication from a particular sensor 66 that a person has entered the particular building space 62 through that access point 64 and may be decremented in response to the controller 24 receiving an indication from a particular sensor 66 that a person has exited the particular building space 62 through that access point 64.

Once a particular counter 68 has reached a particular threshold, the controller 24 may take appropriate action. In some cases, taking action in response to the counter 68 reaching a threshold may include issuing an audio and/or visual warning that current occupancy within a particular building space 62 has reached the threshold for that particular building space 62. Taking action may, for example, include securing the access point 66 for the particular building space 62 such that people are allowed to exit the building space 62 but are not allowed to enter the building space 62 until the occupancy counter 68 for that particular building space 62 drops below the threshold. In some cases, taking action may include increasing an air ventilation rate for that building space 62.

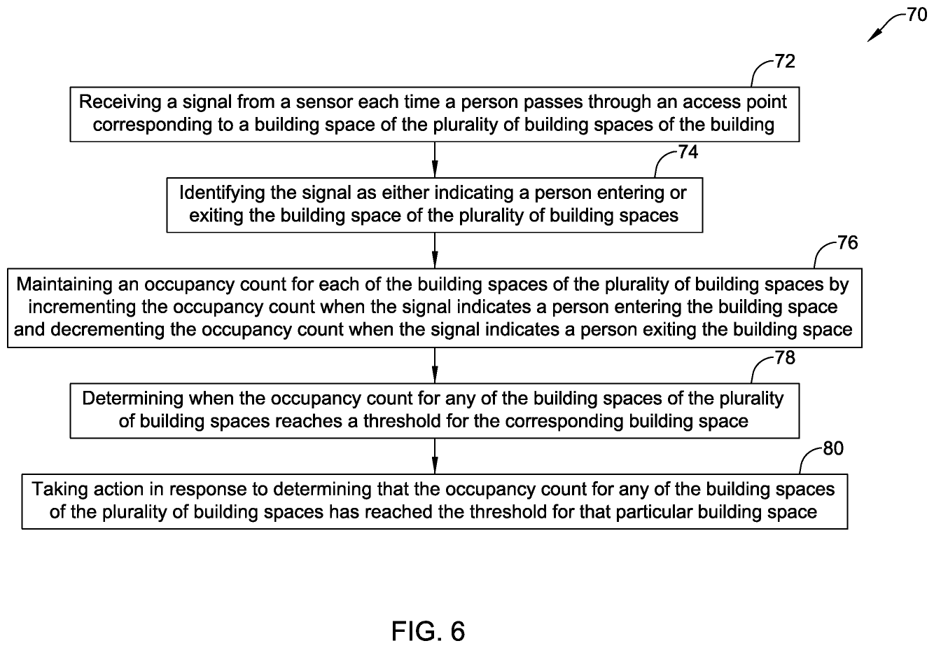

FIG. 6 is a flow diagram showing an illustrative method 70 for tracking occupancy within a building that includes a plurality of building spaces, with each building space including an access point that allows access to the building space and a sensor that provides a signal when a person passes through the access point. A signal is received from a sensor each time a person passes through an access point corresponding to a building space of the plurality of building spaces of the building, as indicated at block 72. The signal is identified as either indicating a person entering or exiting the building space of the plurality of building spaces, as indicated at block 74. An occupancy count is maintained for each of the building spaces of the plurality of building spaces by incrementing the occupancy count when the signal indicates a person entering the building space and decrementing the occupancy count when the signal indicates a person exiting the building space, as indicated at block 76. A determination may be made that the occupancy count for any of the building spaces of the plurality of building spaces has reached a threshold for the corresponding building space, as indicated at block 80.

In response to determining that the occupancy count for any of the building spaces of the plurality of building spaces has reached the threshold for that particular building space, action is taken, as indicated at block 80. Taking action may, for example, include issuing an audio and/or visual warning that current occupancy within a particular building space of the plurality of building spaces has reached the threshold for that particular building space. In some instances, taking action includes securing the access point for the particular building space of the plurality of building spaces such that people are allowed to exit but are not allowed to enter until the occupancy count for that particular building space drops below the threshold. Taking action can additionally or alternatively include increasing an air ventilation rate for the particular building space of the plurality of building spaces.

In some cases, the sensor associated with the access point of at least one of the building spaces of the plurality of building spaces may include a smart floor sensor embedded in a floor covering. The sensor associated with the access point of at least one of the building spaces of the plurality of building spaces may include a passive infrared (PIR) sensor. The sensor associated with the access point of at least one of the building spaces of the plurality of building spaces may include an RFID card reader that can read an access card carried by a person without requiring the person to physically scan the access card to provide frictionless access through the corresponding access point.

The sensor associated with the access point of at least one of the building spaces of the plurality of building spaces may include a video camera having a field of view that includes at least part of the corresponding access point, wherein video analytics are applied to identify the signal from the video camera as either indicating a person entering or exiting the building space of the plurality of building spaces. The video analytics may include identifying biometric identification. The video analytics may include identifying compliance with one or more behavioral thresholds such as but not limited to one or more of a social distancing compliance threshold, a hand hygiene compliance threshold and a Personal Protective Equipment (PPE) compliance threshold.

The access point may, for example, include one of a building entry, an emergency exit, a door or hall to a room or zone, an elevator, and a stair case. At least one of the access points may further include a health screening sensor for performing a health screen of a person passing through the access point, wherein the health screening sensor comprises one or more or a video camera, a thermal camera and a microphone.

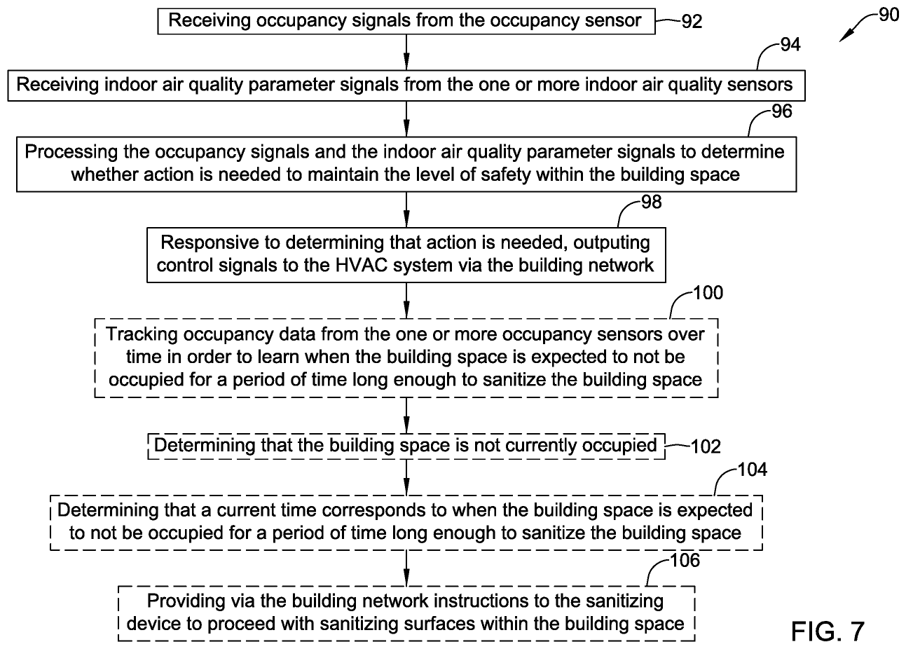

FIG. 7 is a flow diagram showing an illustrative method 90 of maintaining a level of occupant safety within a building having a building space, the building space including an occupancy sensor and one or more air quality sensors, the building space serviced by a heating, ventilating and air conditioning (HVAC) system, each of the sensors and the HVAC system operably coupled with a building network. Occupancy signals are received from the occupancy sensor, as indicated at block 92. Indoor air quality parameter signals are received from the one or more indoor air quality sensors, as indicated at block 94. The occupancy signals and the indoor air quality parameter signals are processed to determine whether action is needed to maintain the level of safety within the building space, as indicated at block 96. Responsive to determining that action is needed, control signals are outputted to the HVAC system via the building network, as indicated at block 98.

In some cases, the building space may be serviced by a sanitizing device. The method 90 may further include tracking occupancy data from the one or more occupancy sensors over time in order to learn when the building space is expected to not be occupied for a period of time long enough to sanitize the building space, as optionally indicated at block 100. A determination may be made that the building space is not currently occupied, as optionally indicated at block 102. A determination may be made that a current time corresponds to when the building space is expected to not be occupied for a period of time long enough to sanitize the building space, as optionally indicated at block 104. In some cases, and as optionally indicated at block 106, instructions may be provided via the building network to the sanitizing device to proceed with sanitizing surfaces within the building space. In some cases, a warning may be issued via the building network that the building space is currently being sanitized.

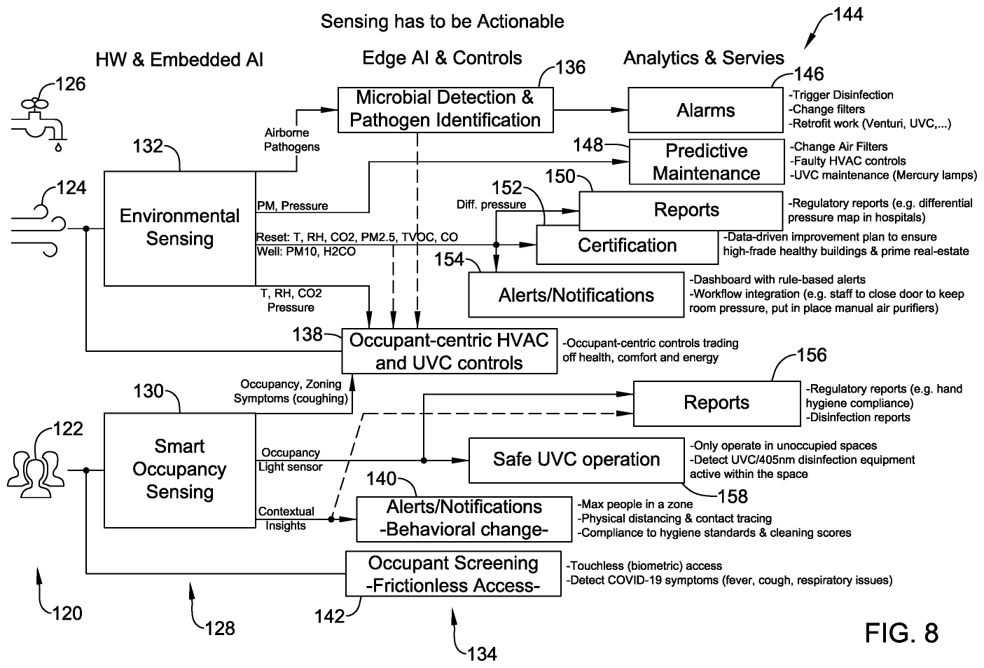

FIG. 8 is a schematic block diagram illustrating relationships between sensing and corresponding actions. On the far left are possible inputs 120. As shown, the possible inputs 120 may include people, as indicated by a people icon 122. The possible inputs 120 may include environmental conditions, as represented by a wind icon 124. In some cases, the possible inputs 120 may include a pathogen, as indicated by the pathogen icon 126. These possible inputs 120 may be sensed via a sensing section 128. The sensing section 128 may include a smart occupancy sensing block 130 and an environmental sensing block 132. Possible outputs from the smart occupancy sensing block 130 may include one or more of occupancy numbers for a zone, as well as possibly detected symptoms within the zone such as coughing. Possible outputs from the smart occupancy sensing block 130 may also include occupancy detection that might cause lighting to be turned on or off, for example. In some cases, a degree of hysteresis or a short delay may be implemented in order to prevent a possible situation in which the lights turn on and off while a person lingers in the doorway, for example, which can be hard on lighting equipment and may be uncomfortable for the person in the doorway. Even if hysteresis is implemented with respect to lighting control, the smart occupancy sensing block 130 may immediately report occupancy.

Possible outputs from the environmental sensing block 132 may include detection of airborne pathogens. Possible outputs from the environmental sensing block 132 may include any of a variety of different air quality parameters such as but not limited to particulate matter (PM), air pressure, temperature, relative humidity, carbon dioxide concentration, PM2.5, total volatile organic compound (TVOC) concentration, carbon monoxide concentration, H2CO (formaldehyde) concentration, and the like.

An actions section 134 includes a number of different possible actions. It will be appreciated that, as shown, the inputs to individual blocks within the actions section 134 (the possible outputs from the sensing section 128) can vary, depending on the specific actions block. For example, the actions section 134 includes a Microbial Detection and Pathogen Identification block 136 that receives sensed data pertaining to sensed airborne pathogens. The actions section 134 includes an Occupant-centric HVAC and UVC Controls block 138. As can be seen, the Occupant-centric HVAC and UVC Controls block 138 can receive a number of different inputs, including occupancy data from the Smart Occupancy Sensing block 130 and a number of air quality parameters from the Environmental Sensing block 132. The actions section 134 also includes an Alerts/Notifications block 140 and an Occupant Screening block 142. The Alerts/Notifications block 140 may output information pertaining to maximum allowed occupancy, physical distancing between people, contact tracing and compliance to hygiene standards. The Occupant Screening block 142 may output information related to touchless or biometric access and detecting symptoms of illness.

An analytics section 144 includes an Alarms block 146 and a Predictive Maintenance block 148. Illustrative outputs from the Alarms block 146 and the Predictive Maintenance block 148 are listed. A Reports block 150 and a Certification block 152 can output regulatory reports and data-driven improvement plans, respectively, as shown. An Alerts/Notifications block 154 can output information to a dashboard. A Safe UVC Operation block 158 confirms that UVC sterilization only occurs in unoccupied spaces.

Having thus described several illustrative embodiments of the present disclosure, those of skill in the art will readily appreciate that yet other embodiments may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A system for reducing the likelihood of disease transmission among occupants of a building that has a plurality of building spaces, the system comprising:
one or more occupancy sensors positioned to detect occupancy within each of the plurality of building spaces, each of the one or more occupancy sensors operably coupled with a building network and providing occupancy signals over the building network;
one or more environmental sensors positioned to detect one or more air quality parameters within at least some of the plurality of building spaces, each of the one or more environmental sensors operably coupled with the building network and providing environmental parameter signals over the building network;
one or more controllable building components for controlling environmental conditions within at least some of the plurality of building spaces to reduce the likelihood of disease transmission among occupants of the building, the one or more controllable building components controllable over the building network;
a controller operably coupled with the building network, wherein the controller is configured to:
receive occupancy signals from the one or more occupancy sensors over the building network;
receive environmental signals from the one or more environmental sensors over the building network;
process the received occupancy signals and the received environmental parameter signals to determine whether action is needed to improve one or more environmental conditions within at least some of the plurality of building spaces to reduce the likelihood of disease transmission among occupants of the building; and
responsive to determining that action is needed, outputting control signals to one or more of controllable building components over the building network to improve one or more environmental conditions within at least some of the plurality of building spaces to reduce the likelihood of disease transmission among occupants of the building.

2. The system of claim 1, wherein the controller is configured to process one or more of the received occupancy signals to identify a measure of compliance of one or more of the occupants of the building with one or more predefined behavioral standards.

3. The system of claim 2, wherein the one or more predefined behavioral standards comprises a social distancing standard.

4. The system of claim 2, wherein the one or more predefined behavioral standards comprises a maximum people per building space standard.

5. The system of claim 2, wherein the one or more predefined behavioral standards comprises a hygiene standard.

6. The system of claim 2, wherein the one or more predefined behavioral standards comprises symptoms standard.

7. The system of claim 2, wherein the one or more predefined behavioral standards comprises a cleaning standard associated with a cleaning crew.

8. The system of claim 1, wherein the controller is configured to process one or more of the received occupancy signals to identify a measure of a total number of occupants within one or more of the plurality of building spaces of the building, and uses the measure of the total number of occupants within one or more of the plurality of building spaces of the building to determine whether action is needed.

9. The system of claim 1, wherein the controller is configured to process one or more of the received environmental parameter signals to identify a measure of compliance with one or more air quality standards.

10. The system of claim 9, wherein the one or more air quality standards comprises a relative humidity standard.

11. The system of claim 9, wherein the one or more air quality standards comprises a temperature standard.

12. The system of claim 9, wherein the one or more air quality standards comprises a CO2 standard.

13. The system of claim 9, wherein the one or more air quality standards comprises one or more of a Particulate Matter (PM) standard, a VOC standard, a CO standard, a H2CO standard, and an airborne pathogen standard.

14. The system of claim 1, wherein the one or more controllable building components comprises an HVAC component.

15. The system of claim 1, wherein the one or more controllable building components comprises an disinfecting component.

16. The system of claim 1, wherein the controller is further configured to determine when action is needed by one or more persons to improve one or more environmental conditions within at least some of the plurality of building spaces to reduce the likelihood of disease transmission among occupants of the building, and provide a notification.

17. A method of maintaining a level of occupant safety within a building having a building space, the building space including an occupancy sensor and one or more air quality sensors, the building space serviced by a heating, ventilating and air conditioning (HVAC) system, each of the sensors and the HVAC system operably coupled with a building network, the method comprising:
receiving occupancy signals from the occupancy sensor;
receiving indoor air quality parameter signals from the one or more indoor air quality sensors;
processing the occupancy signals and the indoor air quality parameter signals to determine whether action is needed to maintain the level of safety within the building space; and
responsive to determining that action is needed, outputting control signals to the HVAC system via the building network.

18. The method of claim 17, wherein the building space is services by a sanitizing device, and the method further comprises:
tracking occupancy data from the one or more occupancy sensors over time in order to learn when the building space is expected to not be occupied for a period of time long enough to sanitize the building space;
determining that the building space is not currently occupied;
determining that a current time corresponds to when the building space is expected to not be occupied for a period of time long enough to sanitize the building space; and
providing via the building network instructions to the sanitizing device to proceed with sanitizing surfaces within the building space.

19. The method of claim 18, further comprising providing via the building network a warning that the building space is currently being sanitized.

20. A system for reducing pathogenic exposure within a building having a plurality of building spaces, the system comprising:
one or more occupancy sensors positioned to detect occupancy within each of the plurality of building spaces, each of the one or more occupancy sensors operably coupled with a building network;
one or more sanitizers, each of the one or more sanitizers positioned to sanitize surfaces within a corresponding one of the plurality of building spaces;
a controller that is operably coupled with the building network such that the controller is configured to:
receive occupancy signals from the one or more occupancy sensors;
process the occupancy signals to determine whether a particular building space is due to be sanitized and is currently available to be sanitized; and
responsive to determining that the particular building space is due to be sanitized and is currently available to be sanitized, outputting appropriate control signals to one or more of the one or more sanitizers to proceed with sanitizing the particular building space.
